(19) United States
(12) Patent Application Publication
(10) Pub. No.: US 2012/0152270 A1
(43) Pub. Date: Jun. 21, 2012

(54) METHOD & APPARATUS FOR APPLYING HAIR ROOT CONCEALER

(76) Inventor: Rita Hazan, New York, NY (US)

(21) Appl. No.: 12/972,094

(22) Filed: Dec. 17, 2010

(51) Int. Cl. A61K 8/58; A61Q 5/00

(52) U.S. Cl. 132/208; 424/47; 8/405

(57) ABSTRACT

The present invention provides an aerosol spray unit 10 containing the hair root concealer composition, having an aerosol dispensing unit 12, which includes a spray nozzle 14 and an inserting tube 16 for insertion directly into the hair roots for applying a hair root concealer composition 18 directly onto the hair roots 20. Aerosol spray unit 10 has an aerosol dispensing unit 12 mounted on top of the unit 10. The short inserting tube 16 is connected to the spray nozzle 14 of dispensing unit 12. Typically, the inserting tube 16 has a length of ¼ inch to 4 inches in order to extend in between the hair roots 20 to apply the concealer onto the hair roots. The hair root concealer is in the form of a composition including butane, propane, ethyl trisiloxane, trimethlysiloxysilicate, methyl trimethicone, titanium dioxide, iron oxide, mica, and hydrofluorocarbon 152A.

### FIELD OF THE INVENTION

The present invention relates to an aerosol unit having a dispensing unit, which includes a spray nozzle and inserting tube, mounted on top of the aerosol unit, in order to easily and cleanly spray the hair root concealer through the inserting tube directly onto the hair roots. The inserting tube extends into the hair roots to dispense the hair root concealer.

### BACKGROUND OF THE INVENTION

Although the prior art discloses a number of applicators discussed below for applying a hair product to the hair using a brush, it does not disclose an aerosol spray unit with a spray nozzle and short inserting tube attached to the nozzle for spraying a hair root concealer composition directly in between the hair roots.

European Patent Application No. WO 2006/024365 is owned by Henkel of Germany. It is entitled “Applicator” and discloses the concept of a device for applying a hair product, such as a hair dye, to the base of the hair using a brush. It also includes a nozzle, but no inserting tube for inserting into the hair roots. As seen in the drawing on the first page of the application, the applicator (1) is for applying a hair treatment agent, in particular for redying renewable hairs, to a hair-roots area. It uses an application nozzle (3) for the hair treatment agent and a closing element (5) for the application nozzle (3). The aim is to design an applicator which is usable by a lay person, makes it possible to carry out the accurate hair-treatment agent dosing, prevents from delivering other treatment products during distribution and which is easily mountable. For this purpose, the distributor element (4) has operating and resting positions. The closing element (5) has closed and open positions, wherein the distributor element (4) is mechanically coupled with the closing element (5) in such a way that said closing element (5) is in the closed position when the distributor element (4) is in the operating position.

The applicator has a squeeze bottle (13) and the dye is squeezed or forced through the nozzle/tube (3, 15) to the brush (14) at the tip. Therefore, this patent does not disclose an aerosol nozzle and inserting tube for easily spraying a concealer on the hair roots, as in the present invention, since it discloses a squeezing device.

The website of Jerome Russell discloses aerosol spray products. It has a regular spray nozzle, with no tube attached. In fact, the website states that you hold the can 12 to 18 inches away from the hair scalp. Therefore, Russell does not disclose a nozzle having a hollow tube connected to it for direct contact with the hair roots, as in the present invention.

U.S. Pat. No. 6,009,881 entitled “Applicator Device For A Hair Product And Receptacle Equipped With Such A Device” also discloses the concept of a device for applying a hair product, such as a hair dye, to the base of the hair. It includes an enclosure having tapered ends which are able to separate the hair on either side of a part. Its end piece is designed to be mounted on a receptacle containing the product, optionally via an applicator end-piece surmounting the receptacle in such a way as to enable the product to be applied in a localized manner. As seen in FIGS. 1A and 1B, the device (1) comprises an enclosure (2) in the general shape of a spout. In this embodiment, the tapering end (4) serves to separate the hair on either side of a part. The applicator end-piece (105) is for applying the product. The free edge of the wall (3) is for spreading the product on the base of the hair near the part. In order to use such an assembly, the user inverts the receptacle and places the end (4) on the hair near the forehead for example, keeping the edge (9) of the application surface in contact with the head. The walls of the receptacle are squeezed so as to force some of the product out through the orifice (108). The face (3) closes the pathway and its free edge spreads the product over the base of the hair. The user then takes the device back to another starting point which is offset sideways from the first starting point, and performs the same movement again in order to treat an adjacent zone. This is continued until all the roots have been completely treated.

U.S. Pat. No. 4,209,027 entitled “Hair Treatment Devices And Packaging Therefor” also discloses the concept of a device for applying a hair product, such as a hair dye, to the base of the hair. It also includes an enclosure having tapered ends which are able to separate the hair on either side of a part. As seen in FIGS. 1 and 3, a hair treatment device (1) is secured in liquid tight relation to a squeeze bottle (2) to form a combination that may be held in the hand (3) of a cosmetologist or other operator, the upside-down or working position for sectioning and parting the hair of a customer for treatment and supplying one or more treating liquids to and spreading them on selectively sectioned strands or tresses.

U.S. Pat. No. 4,211,247 entitled “Devices For Use In Selectively Altering Hair Color” also discloses the concept of a device for applying a hair product, such as a hair dye, to the base of the hair. It also includes an enclosure with an end that is able to separate the hair on either side of a part. As seen in FIGS. 1 and 2, the hair parting means (24) and a liquid spreading means (32) are attached to this end and it enables the user to accurately apply and spread the hair dye on selectively sectioned strands or tresses.

### OBJECTS OF THE PRESENT INVENTION

None of the aforementioned prior art patents teach or disclose an aerosol spray unit 10 containing the hair root concealer composition having an aerosol dispensing unit 12, which includes a nozzle 14 and a short inserting tube 16, for applying a hair root concealer composition 18 directly onto the hair roots 20.

Accordingly, it is an object of the present invention to provide an aerosol spray unit 10, having an aerosol dispensing unit 12, including a spray nozzle 14 and an inserting tube 16 for applying a hair root concealer composition 18 directly onto the hair roots 20.

Another object of the present invention is to provide a spray nozzle 14 connected to the dispensing unit, wherein the inserting tube 16 typically has a length of ¼ inch to 4 inches in order to extend between the hair roots 20 to directly apply the hair root concealer onto the base of the hair roots.

### SUMMARY OF THE INVENTION

The present invention provides an aerosol spray unit 10 containing the hair root concealer composition, having an aerosol dispensing unit 12, which includes a spray nozzle 14 and an inserting tube 16 for insertion directly into the hair roots for applying a hair root concealer composition 18 directly onto the hair roots 20. Aerosol spray unit 10 has an aerosol dispensing unit 12 mounted on top of the unit 10. The short inserting tube 16 is connected to the spray nozzle 14 of dispensing unit 12. Typically, the inserting tube 16 has a length of ¼ inch to 4 inches in order to extend in between the hair roots 20 to apply the concealer onto the hair roots. The hair root concealer is in the form of a composition including butane, propane, ethyl trisiloxane, trimethlysiloxysilicate, methyl trimethicone, titanium dioxide, iron oxide, mica, and hydrofluorocarbon 152A.

### BRIEF DESCRIPTION OF THE DRAWINGS

Further objects, features and advantages of the present invention will become apparent upon the consideration of the following detailed description of the presently preferred embodiment when taken in conjunction with the accompanying drawings, wherein:

FIG. 1 is a perspective view of an aerosol spray unit, nozzle, and inserting tube for applying hair root concealer to the hair roots; and

FIG. 2 is a perspective view of the details of the spray unit, nozzle, and inserting tube, showing the aerosol unit applying the hair root concealer to the hair roots.

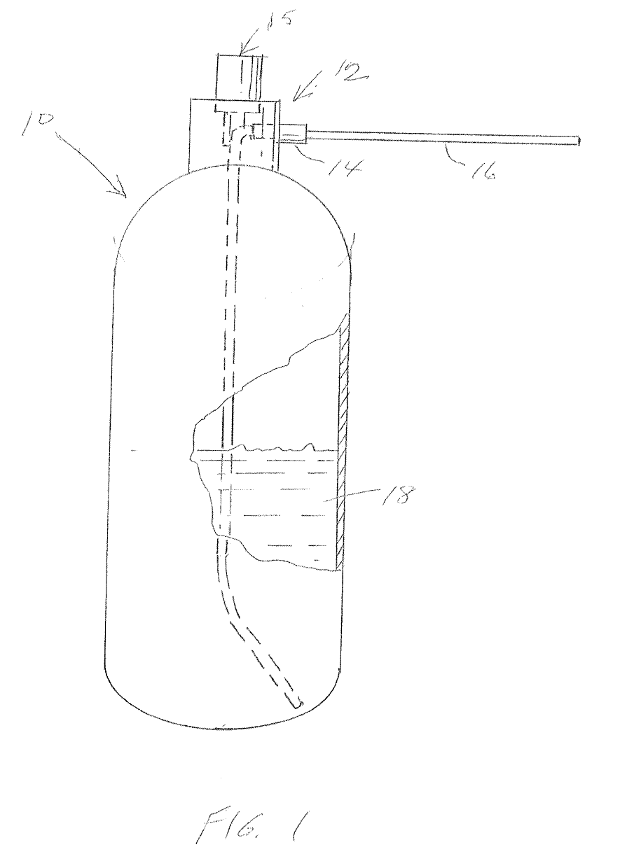

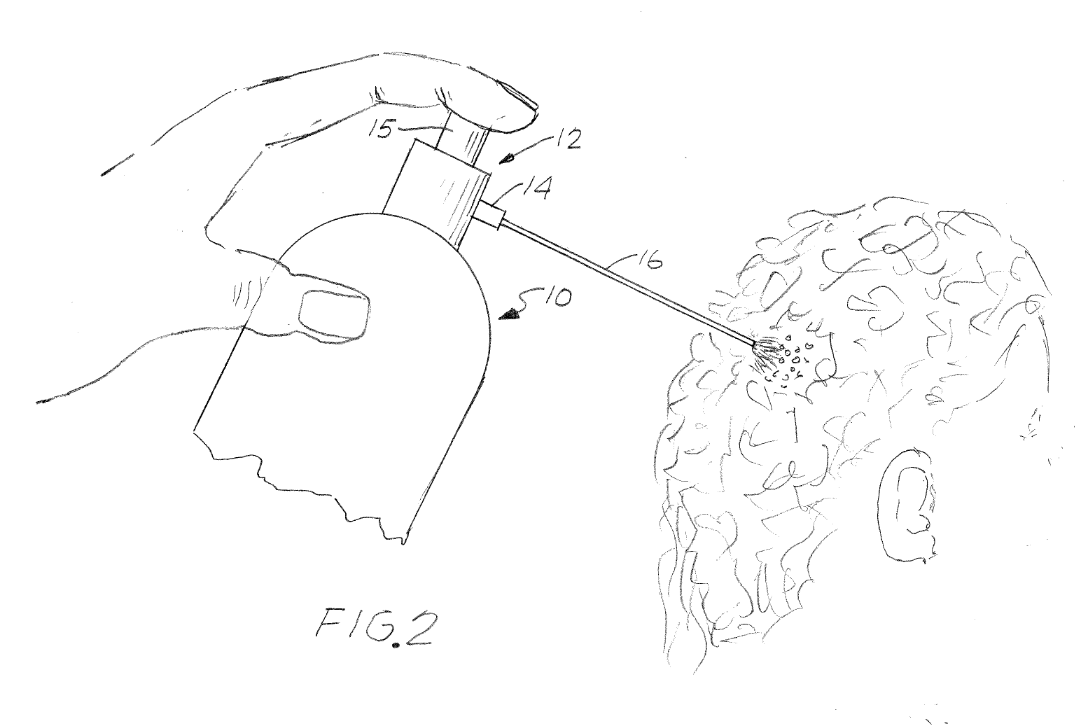

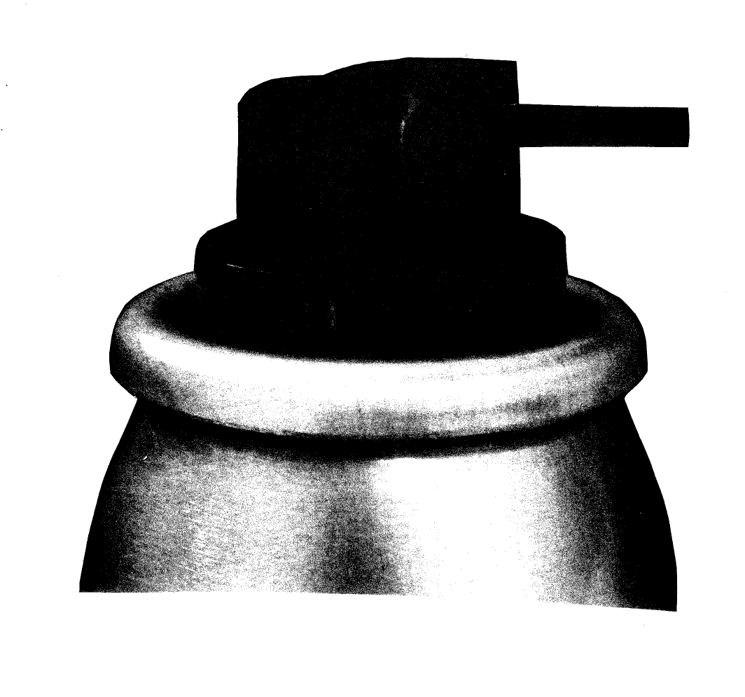

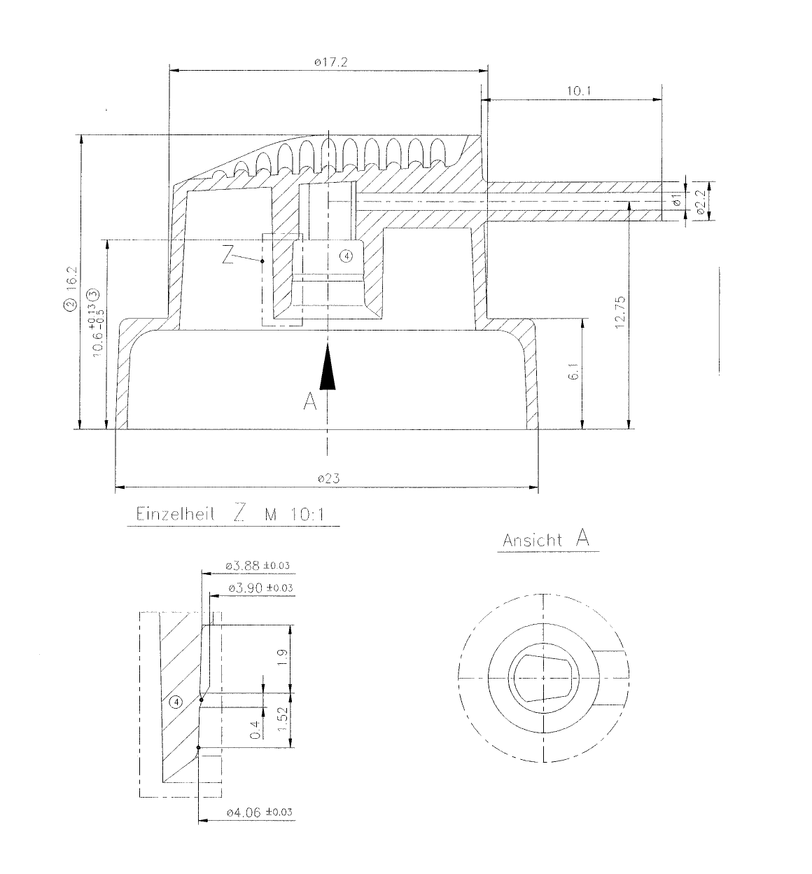

### DETAILED DESCRIPTION OF THE PREFERRED EMBODIMENT

The present invention provides an aerosol spray unit 10, having an aerosol dispensing unit 12, which includes a spray nozzle 14 and an inserting tube 16 for spraying the hair root concealer composition 18 between the hair roots 20. Aerosol spray unit 10 has an aerosol dispensing unit 12 mounted on the top of the unit 10. The short inserting tube 16 is connected to the spray nozzle 14. Typically, the inserting tube 16 has a length of ¼ inch to 4 inches in order to extend between the hair roots 20 to apply the concealer 18, which is a composition including butane, propane, ethyl trisiloxane, trimethlysiloxysilicate, methyl trimethicone, titanium dioxide, iron oxide, mica, and hydrofluorocarbon 152A. In addition, the preferred internal diameter of tube 16 is one millimeter, however, the range can be 1 to 4 millimeters.

The present invention also provides a method for spraying hair root concealer directly onto the hair roots, comprising the steps of using an aerosol spray unit 10 having a spray nozzle 14 and inserting tube 16; then placing the inserting tube 16 into the hair so the end of the inserting tube 16 is between the hair roots; then actuating the top of the spray unit 10 for spraying hair root concealer through the inserting tube 16 directly onto the hair roots. Then the inserting tube 16 is moved to another position between the hair roots of the user; and then the top of the spray unit 10 is actuated again for spraying hair root concealer through the inserting tube 16 directly onto the hair roots.

The present invention also provides a hair root concealer composition 18 for applying to the hair roots using an aerosol spray unit. The composition includes butane and propane as aerosol propellants, as well as butane, propane, ethyl trisiloxane, trimethlysiloxysilicate, methyl trimethicone, titanium dioxide, iron oxide, mica, and hydrofluorocarbon 152A.

### OPERATION OF THE PRESENT INVENTION

To apply the hair root concealer onto the base of the hair roots 20, the user inserts the inserting tube 16 directly into the hair roots. The spray unit is pressed downwardly and the hair root concealer composition 18 passes through inserting tube 16 and is deposited directly onto the hair roots 20. The inserting tube 16 has a length of ¼ inch to 4 inches in order to extend between the hair roots 20 to apply the concealer, which is in the form of a composition including butane, propane, ethyl trisiloxane, trimethlysiloxysilicate, methyl trimethicone, titanium dioxide, iron oxide, mica, and hydrofluorocarbon 152A.

Then, the inserting tube 16 is moved to another position between the hair roots and the process is repeated.

### ADVANTAGES OF THE PRESENT INVENTION

Accordingly, an advantage of the present invention that it provides an aerosol spray unit 10, having an aerosol dispensing unit 12, which includes a spray nozzle 14 and inserting tube 16 for applying a hair root concealer composition 18 directly onto the hair roots 20.

Another advantage of the present invention is that it provides a short inserting tube 16 connected to the spray nozzle 14, wherein the inserting tube 16 has a length of ¼ inch to 4 inches in order to extend between the hair roots 20 to directly apply the concealer onto the base of the hair roots.

A latitude of modification, change and substitution is intended in the foregoing disclosure, and in some instances, some features of the invention will be employed without a corresponding use of other features. Accordingly, it is appropriate that the appended claims be construed broadly and in a manner consistent with the spirit and scope of the invention herein.

What is claimed is:

1) An aerosol spray unit for applying hair root concealer directly onto the hair roots, comprising:
a) an aerosol dispensing unit 12 located at the top of the spray unit 10; said spray unit containing the hair root concealer composition, including butane and propane as propellants;
b) a spray nozzle 14 is connected to the spray unit 10 for spraying the hair root concealer composition 18 onto the hair roots 20; and
c) a short inserting tube 16 is connected to the spray nozzle 14 with the inserting tube 16 for extending between the hair roots 20 to apply the concealer 18 directly onto the hair roots.

2) An aerosol spray unit in accordance with claim 1, wherein said inserting tube 16 is between ¼ inch to 4 inches in length for extending into the hair roots, and the internal diameter of said inserting tube is one millimeter to 3 millimeters.

3) A method for spraying hair root concealer directly onto the hair roots, comprising the steps of:
a) using an aerosol spray unit 10 having a spray nozzle 14 and inserting tube 16;
b) placing the inserting tube 16 into the hair so the end of the inserting tube is between the hair roots;
c) actuating said spray unit for spraying hair root concealer through said inserting tube 16 directly onto the hair roots;
d) moving said inserting tube 16 to another position between the hair roots of the user; and
e) again actuating said spray unit for spraying hair root concealer through said inserting tube 16 directly onto the hair roots.

4) A hair root concealer for applying to the hair roots using an aerosol spray unit, comprising a composition butane, propane, ethyl trisiloxane, trimethlysiloxysilicate, methyl trimethicone, titanium dioxide, iron oxide, mica, and hydrofluorocarbon 152A.
